(19) United States
(12) Patent Application Publication
(10) Pub. No.: US 2022/0152153 A1
(43) Pub. Date: May 19, 2022

(54) STABILIZED AFGF COMPOSITIONS

(71) Applicant: EUSOL BIOTECH CO., LTD., Taipei (TW)

(72) Inventor: Jin-Ding HUANG, Taipei City (TW)

(73) Assignee: EUSOL BIOTECH CO., LTD., Taipei (TW)

(21) Appl. No.: 17/526,203

(22) Filed: Nov. 15, 2021

Related U.S. Application Data
(60) Provisional application No. 63/114,044, filed on Nov. 16, 2020.

(51) Int. Cl. A61K 38/18; A61K 47/12

(52) U.S. Cl. CPC A61K 38/1825; A61K 47/12

(57) ABSTRACT

A stabilized aFGF composition or a method for stabilization of the pharmaceutical composition comprising aFGF are provided. The composition comprises aFGF, a citric acid compound and other excipients. The method for improving stability of aFGF in the form of a liquid formulation or a lyophilized formulation so as to increase storage stability is also provided.

### CROSS REFERENCE

This Non-provisional application claims the priority under 35 U.S.C. § 119(a) on U.S. Patent Provisional Application No. 63/114,044 filed on Nov. 16, 2020, the entire contents of which are hereby incorporated by reference.

### FIELD OF THE INVENTION

The present invention relates to a stabilized aFGF composition or a method for stabilization of the pharmaceutical composition comprising aFGF.

### BACKGROUND OF THE INVENTION

Acidic fibroblast growth factor (aFGF, also called FGF1) was originally isolated as single chain proteins from neural tissue, including whole brain and hypothalamus. It is known that aFGF can promote the proliferation and differentiation of various cell types in vitro, and the applications thereof (such as wound healing, hair growth and neuron growth) have been developed in the pharmaceutical field. So far, AiFuJiFu, which has been approved for the treatment of burn and other skin injuries in China since 2006, is the only one recombinant human aFGF (rhaFGF) commercial drug product on market. AiFuJiFu is composed of lyophilized powder containing rhaFGF, human albumin, mannitol and phosphate buffer, and reconstitute for topical spray.

ES135, an aFGF variant, is under phase III clinical trial for treating spinal cord injury by Eusol Biotech in Taiwan (ClinicalTrials.gov Identifier: NCT03229031). Phosphate buffer is also used in the current formulation of ES135. However, the stability test for the formulation of ES135 showed that drug substance was precipitated at 25° C. for 1 month (accompanied by decreasing protein concentration), and the purity of ES135 measured in HPIEC also reduced about fifty percentage. Since the formulation of ES135 is unstable at room temperature, its storage temperature should be strictly at −70° C. in order to keep long term stability, which would cause high-cost cold chain shipment. Given the above, stability is the major issue of current formulation of ES135.

In order to improve stability of current formulation, the applicant tried to find out more effective components or composition to stabilize ES135 and rise its storage temperature. Previous study revealed that high concentration of NaCl could slow down the precipitation of ES135. However, high concentration of NaCl in drug product could cause adverse effects if the osmolality is higher than physical limit. Moreover, ES135 in NaCl solution had limited stability since it needs to be kept at −20° C. (storage temperature), which is still not suitable for commercialization.

On the other hand, deamidation of ES135 was observed during the stability test, and the deamidated ES135 could increase more than 15% at room temperature for 1 month even if the formulation contains high concentration of NaCl. The fast growth of deamidation impurity also limited the storage temperature and not suitable for commercialization.

Accordingly, there is still a need for aFGF (particularly the variant ES135) formulations with improved stability.

### SUMMARY OF THE INVENTION

It was unexpectedly found in the present invention that a citrate buffer comprising a citric acid compound provides an efficacy in improving the stability of pharmaceutical compositions comprising aFGF, particularly ES135.

Accordingly, one aspect of the present invention is to provide a pharmaceutical composition comprising aFGF and a citric acid compound.

In one embodiment of the invention, the citric acid compound is citric acid or isocitric acid.

In one embodiment of the invention, the pharmaceutical composition is in the form of a liquid or a lyophilized formulation.

In one embodiment of the invention, the concentration for the citric acid compound in the liquid formulation ranges from 5 mM to 75 mM.

In one embodiment of the invention, the liquid formulation has a pH value within the range of pH 5.8 to pH 7.0.

In one embodiment of the invention, aFGF in the pharmaceutical composition is the protein having an amino acid sequence at least 70%, 75%, 80%, 85%, 90%, 95%, or 98% identical to the amino acid sequence of SEQ ID NO: 1.

In one embodiment of the invention, the pharmaceutical composition is administered subcutaneously, topically, intranasally, intravenously, intramuscularly, intraneurally, intraperitoneally, intracerebroventricularly or intrathecally.

In one embodiment of the invention, the pharmaceutical composition is in the form of a lyophilized formulation.

In one embodiment of the invention, the pharmaceutical composition further comprising mannitol, sugar, or the combination thereof.

In one embodiment of the invention, the sugar is selected from the group consisting of trehalose, sucrose, and the combination thereof.

Another aspect of the present invention is to provide a new use of a citrate buffer in improving the stability of pharmaceutical compositions.

A further aspect of the present invention is to provide a method for stabilization of a pharmaceutical composition comprising aFGF, particularly ES135, comprising mixing aFGF with a citrate buffer to obtain a liquid formulation, and optionally lyophilizing the liquid formulation.

It is to be understood that both the foregoing general description and the following detailed description are exemplary and explanatory only and are not restrictive of the invention.

### BRIEF DESCRIPTION OF THE DRAWINGS

FIG. 1 shows the result of UV360 absorption in stability test on ES135 dissolved in 3 different buffer systems (5 mM) with different NaCl concentrations on Day 3.

FIG. 2 shows the result of UV360 absorption in stability test on ES135 dissolved in 3 different buffer systems (20 mM) with different NaCl concentrations on Day 3.

FIG. 3 shows the result of UV360 absorption in accelerated stability test on ES135 dissolved in 3 different buffer systems (20 mM).

FIG. 4 shows the result of deamidation level in stability test on ES135 dissolved in 3 different buffer systems (5 or 20 mM) with different pH value on Day 3.

FIG. 5 shows the result of deamidation level in stability test on ES135 dissolved in 2 different buffer systems (20 mM phosphate or 30 mM citrate) from 0 to 90 days.

FIG. 6 shows the result of UV360 absorption in stability test on ES135 dissolved in 4 salt/buffer solution with different concentrations on Day 3.

FIG. 7 shows the result of UV360 absorption in accelerated stability test on ES135 dissolved in 10 mM citrate buffer with different pH values.

FIG. 8 shows the comparison of melting temperature in thermal shift assay on ES135 dissolved in citrate buffer and phosphate buffer.

### DETAILED DESCRIPTION OF THE INVENTION

Provided herein are pharmaceutical compositions comprising aFGF and a citric acid compound. Based on the studies as described herein, the inventors have shown that citric acid compound has improved effects in stabilizing the pharmaceutical compositions comprising aFGF.

The following abbreviations are used herein:
SEC: Size Exclusion Chromatography
RP-HPLC: Reversed Phase High Performance Liquid Chromatography
HPIEC: High Performance Ion Exchange Chromatography
aFGF: acidic Human Fibroblast Growth Factor
SDS PAGE: Sodium Dodecyl Sulfate Polyacrylamide Gel Electrophoresis
mM: millimolar (10⁻³ mol/L)

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

As used herein, the article “a” or “an” means one or more than one (that is, at least one) of the grammatical object of the article, unless otherwise made clear in the specific use of the article in only a singular sense.

As used herein, the term “aFGF” refers to a naturally-occurring, isolated, recombinant, or synthetically-produced aFGF which includes allelic variants, species homologs, or any modified peptide thereof. The modified peptide may be obtained such as by one or more deletions, insertions, substitutions or combinations thereof in the aFGF as defined above. In one embodiment of the invention, the modified aFGF is a peptide comprising a native human aFGF (154 amino acids in length) shortened by a deletion of 20 amino acids from N-terminal, and an addition of alanine before the shortened native human aFGF. For example, the modified aFGF may be a peptide consisting of the amino acid sequence of SEQ ID NO:1 (also called ES135), as described in U.S. Pat. No. 7,956,033 (U.S. patent application Ser. No. 12/482,041), the content of which is hereby incorporated by reference herein in its entirety.

According to the invention, the aFGF is a protein consisting of the amino acid sequence of SEQ ID NO:1 below:

Ala Asn Tyr Lys Lys Pro Lys Leu Leu Tyr Cys Ser
Asn Gly Gly His Phe Leu Arg Ile Leu Pro Asp Gly
Thr Val Asp Gly Thr Arg Asp Arg Ser Asp Gln His
Ile Gln Leu Gln Leu Ser Ala Glu Ser Val Gly Glu
Val Tyr Ile Lys Ser Thr Glu Thr Gly Gln Tyr Leu
Ala Met Asp Thr Asp Gly Leu Leu Tyr Gly Ser Gln
Thr Pro Asn Glu Glu Cys Leu Phe Leu Glu Arg Leu
Glu Glu Asn His Tyr Asn Thr Tyr Ile Ser Lys Lys
His Ala Glu Lys Asn Trp Phe Val Gly Leu Lys Lys
Asn Gly Ser Cys Lys Arg Gly Pro Arg Thr His Tyr
Gly Gln Lys Ala Ile Leu Phe Leu Pro Leu Pro Val
Ser Ser Asp.

In some embodiments, the sequence of the aFGF is at least 70%, 75%, 80%, 85%, 90%, 95%, 98%, or 100% identical to the amino acid sequence of SEQ ID NO: 1. In some embodiments, the amino acid sequence of SEQ NO:1 has one or more modifications. For example, the amino acid sequence of SEQ NO:1 has an N-terminal phosphogluconoylation or gluconoylation as disclosed in U.S. Pat. No. 9,567,385 (U.S. patent application Ser. No. 14/508,118), the content of which is hereby incorporated by reference herein in its entirety.

As used herein, the term “pharmaceutical composition” as used herein refers to a finished dosage formulation, which contains active ingredient (aFGF as an example) and which is in a form in which it can be marketed for use. The pharmaceutical composition could be in liquid or lyophilized form.

As used herein, the term “citric acid compound” as used herein refers to citric acid (2-Hydroxypropane-1,2,3-tricarboxylic acid), isocitric acid (1-Hydroxypropane-1,2,3-tricarboxylic acid), the salt of citric acid (such as sodium dihydrogen citrate, disodium hydrogen citrate, trisodium citrate, potassium dihydrogen citrate, dipotassium hydrogen citrate tripotassium citrate, or the hydrate form thereof) or isocitric acid (such as sodium dihydrogen isocitrate, disodium hydrogen isocitrate, trisodium isocitrate, potassium dihydrogen isocitrate, dipotassium hydrogen isocitrate tripotassium isocitrate, or the hydrate form thereof), or the combinations thereof. The isocitric acid as defined above can be one of the four stereoisomer, including D-Threo-Isocitric acid (i.e., (1R,2S)-1-hydroxypropane-1,2,3-tricarboxylic acid), L-erythro-isocitric acid (i.e., (1R,2R)-1-hydroxypropane-1,2,3-tricarboxylic acid), L-Threo-isocitric acid (i.e., (1 S,2R)-1-hydroxypropane-1,2,3-tricarboxylic acid), D-erythro-Isocitric acid ((1 S,2S)-1-hydroxypropane-1,2,3-tricarboxylic acid), or the combinations thereof. Preferably, the citric acid compound is citric acid or isocitric acid.

As used herein, the term “liquid” with regard to pharmaceutical compositions comprising aFGF is intended to include the term “aqueous.” The term “lyophilize” or “lyophilized” with regard to pharmaceutical compositions comprising aFGF is intended to refer to freeze drying under reduced pressure of a plurality of vials, each containing a unit dose of aFGF formulation of the present invention therein. Lyophilizers, which perform the above described lyophilization, are commercially available and readily operable by those skilled in the art.

The turbidity test method, UV 360 nm detection, was applied to measure the protein precipitation. The ES135 in some formulations didn't precipitate in a few days; therefore, an accelerated method was also developed for determining the formulation stability rapidly. In the accelerated method, the 96 well plate was placed in spectrophotometer and heated to specific temperatures step by step, the temperature at which ES135 started to precipitate could reflect the ES135 stability in formulations.

HPIEC was adopted to determine the amount of deamidation of ES135. Previous study showed one peak on HPIEC continued growing during the stability study, then the mass spectrum identified the new deamidation product. The HPIEC method was the main method to measure the deamidation impurity in this study.

One embodiment of the invention is a pharmaceutical composition comprises aFGF and a citric acid compound, which provides an improved stability.

In another embodiment of the invention, the pharmaceutical composition with improved stability comprises aFGF and a citric acid compound. The citric acid compound is selected from the group consisting of citric acid and isocitric acid. More preferably, the citric acid compound is citric acid. Citric acid compound (especially citric acid) can stabilize ES135 by preventing precipitation. The tests for evaluating the effect of salt (as buffer system) on formulation stability were described below.

In a further embodiment of the invention, the method for stabilization of a pharmaceutical composition comprising aFGF, particularly ES135, comprising mixing aFGF with a citrate buffer to obtain a liquid formulation, and optionally lyophilizing the liquid formulation.

In other words, the present invention provides a new use of a citrate buffer in improving the stability of pharmaceutical compositions comprising aFGF.

According to the invention, the pharmaceutical composition may be prepared in the form of a liquid formulation. In some embodiments, the range of the concentration for the citric acid compound in the liquid formulation is 5 mM to 75 mM; preferably 5 mM to 20 mM. In one embodiment of the invention, the concentration of the critic acid compound is about 5 mM.

In some embodiments, the pH range of the liquid formulation is pH 5.8-7.0. Suitable pH's include about pH 5.8, about pH 5.9, about pH 6.0, about pH 6.1, about pH 6.2, about pH 6.3, about pH 6.4, about pH 6.5, about pH 6.6, about pH 6.7, about pH 6.8, about PH 6.9, and about pH 7.0. The suitable pH ranges are pH 5.9-7.0; pH 6.0-7.0; pH 6.1-7.0; pH 6.2-7.0; pH 6.3-7.0; pH 6.4-7.0; pH 6.5-7.0; pH 6.6-7.0; pH 6.7-7.0; pH 6.5-6.9; and pH 6.6-6.8. Most preferably, the pH range is pH 6.6-6.8.

In some embodiments of the invention, aFGF may be ES135 consisting of the amino acid sequence of SEQ ID NO: 1, or either of the proteins having an amino acid sequence being at least 70%, 75%, 80%, 85%, 90%, 95%, or 98% identical to the amino acid sequence of SEQ ID NO: 1.

In some embodiments of the invention, the pharmaceutical composition is in the form of a lyophilized formulation. Specifically, the lyophilized formulation comprises a citric acid compound and aFGF. In some embodiments, the lyophilized formulation further comprises mannitol, sugar, or the combinations thereof. Preferably, the sugar is selected from the group consisting of trehalose, sucrose, and the combinations thereof.

According to the invention, ES135 may be constituted into any form suitable for the mode of administration selected. Preferably, ES135 is administered subcutaneously, topically, intranasally, intravenously, intramuscularly, intraneurally, intraperitoneally, intracerebroventricularly or intrathecally. More preferably, ES135 is administered intrathecally.

Methods

The detailed analytical methods utilized in this invention are shown in Table 1 below.

TABLE 1
The analytical methods adopted in this invention

| | Analytical methods | Functions | Descriptions |
|---|---|---|---|
| 1 | UV 360 nm Method | Turbidity | Detect the precipitation of ES135 |
| | | | in liquid formulation |
| 2 | HPIEC Method | Deamidation | Measure the deamidation level of |
| | | | ES135 |
| 3 | UV 360 nm Method | Turbidity | Detect the precipitation of ES135 |
| | (Accelerated method) | | in liquid formulation with |
| | | | increasing temperature |
| 4 | Thermal Shift Assay | Fluorescent | Determine the melting |
| | (TSA) | | temperature of ES135 in liquid |
| | | | formulation |

UV 360 nm Method

The aggregation of protein was measured by the degree of light scattering at 360 nm using Epoch™ 2 microplate spectrophotometer (BioTek). Transfer 300 μl of sample solutions into 96 well plate and the change in optical density at 360 nm over time was measured at specified temperatures over time point. The aggregated analytes formation was irreversible in this period. The absorbance was correlated to the turbidimetric level which was increased when protein precipitated in solution. Their first measurement (O.D.(T0)) was subtracted in order to eliminate background absorbance of the samples.

HPIEC Method

The HPIEC method was used to determine the deamination level of test samples. The weak cation exchange column was used to separate the impurity according to the net changes and the MES buffer with 1M NaCl was selected for eluting the analytes. The samples solution were transfer to glass vial and applied 100 μl into the HPLC system. The detail of the HPIEC method was listed in Table 2.

TABLE 2
HPIEC method

| Solvent A | MES (2-(N-morpholino) ethanesulfonic acid) buffer | |
|---|---|---|
| Solvent B | MES buffer with 1M NaCl | |
| Column | Weak cation-exchange column, Analytical, 4 × 250 mm | |
| Injection volume | 100 | μl |
| Run time | 20 | min |
| Wavelength | 280 | nm |
| Flow rate | 0.5 | ml/min |

| | |
|---|---|
| Mobile phase | 55% Solvent A/45% Solvent B |

Thermal Shift Assay

Thermal shift assay (TSA) with fluorescence of 465 nm excitation and 580 nm emission wavelength was utilized to examine the melting temperature (Tm) of ES135 in various formulations. TSA method was used to compare the effectiveness of buffer systems.

The melting temperature (Tm) of the protein was determined using the maximal change of the fluorescence shift in the first derivative (dRFU/dT) curve of temperature (T) to fluorescent (RFU) using LightCycler®480 II. 20 μl of sample solutions containing SYPRO Orange dye (Sigma-Aldrich, Cat #55692) were transfer into the LightCycler®480 II proprietary 96 well plate (Roche, Cat #04729692001) and treated with a rapid escalation of temperature from 25° C. to 95° C. The inactive aqueous form of SYPRO Orange dye binds to the thermally exposed hydrophobic regions of the protein in order to emit fluorescence. A protein with higher Tm is considered a better stability. Buffer groups were analyzed additionally to exclude non-proteins related signals. The detail of TSA was listed in Table 3.

TABLE 3
Thermal Shift Assay

| Citrate buffer | 30 mM Citrate, 110 mM NaCl, pH 6.5 | |
|---|---|---|
| Tested volume | 20 | μl |
| Run time | 30 | min |

| | |
|---|---|
| Wavelength | Excitation 466 nm/Emission 580 nm |
| Ramp rate | 0.05° C./second; 25° C. to 95° C. |
| Detection rate | Continuous |

Lyophilization of aFGF Formulation

The lyophilizer could remove the water from the samples and provide better stability. Transfer 1 ml of samples solution into 2 ml glass vials and cooled at −40° C. for 30 minutes. The frozen sample experience −15° C. for annealing, and cool back to −40° C. before evacuation. The lyophilization program was listed in Table 4. After the completion of the program, fill the lyophilization chamber with nitrogen gas and seal the stopper under the surrounding of nitrogen. The test samples were dried and fill of nitrogen.

TABLE 4
Lyophilization program

| Sequence | Temperature | | Time | Vacuum |
|---|---|---|---|---|
| 1 | −40° C. Pre-cooling | | 30 min | None |
| 2 | −15° C. (annealing) | | 180 min | None |
| 3 | −40° C. Pre-cooling | | 60 min | None |
| 4 | −40° | C. | 10 min | <0.2 torr |
| 5 | −40 to −37° | C. | 10 min | <0.2 torr |
| 6 | −37° | C. | 1440 min | <0.2 torr |
| 7 | −37 to 20° | C. | 570 min | <0.2 torr |
| 8 | 20° | C. | 480 min | <0.2 torr |

### EXAMPLES

The present invention is more specifically explained by the following examples. However, it should be noted that the present invention is not limited to these examples in any manner.

### Example 1: Effect of Different Salts on Protein Precipitation

Test 1 was designed to evaluate different buffer (phosphate, histidine, and citrate) with NaCl concentrations from 0% to 0.8%, and Test 2 was performed under a range of buffers and NaCl concentrations to compare the stability of ES135 in different salts. The test samples were listed in Table 5, and UV 360 nm absorption and HPIEC were applied to determine the stability of ES135. An accelerated method was performed on samples by placing 96 wells in ELISA reader with a temperature gradient from 25° C. to 65° C., and it could differentiate the stability of test samples in hours. The buffers and their components used in Test 1 and Test 2 were summarized in Table 5.

TABLE 5
Effects of salt on protein precipitation
Test 1

| Factors | Buffer | NaCl |
|---|---|---|
| | 5 mM phosphate | 0% |
| | 20 mM phosphate | 0.1% |
| | 5 mM histidine | 0.4% |
| | 20 mM histidine | 0.8% |
| | 5 mM citrate | — |
| | 20 mM citrate | — |

Total 6 buffers * 4 NaCl concentrations = 24 compositions
Testing methods:
1. UV360 absorption at room temperature on Day 3
2. Apply HPIEC analysis after place at room temperature for 3 days
3. Accelerated method (20 mM buffers, 0.8% NaCl)

Test 2

| Factors | Buffer or salt | Buffer/salt concentrations |
|---|---|---|
| | NaCl | 0, 25, 74, 151, 450 mM |
| | Phosphate | 0, 12, 37, 75, 225 mM |
| | Histidine | 0, 8, 25, 50, 150 mM |
| | Citrate | 0, 4, 12, 24, 75 mM |

Testing methods:
1. UV360 absorption at room temperature on Day 3
2. Apply HPIEC analysis after place at room temperature for 3 days

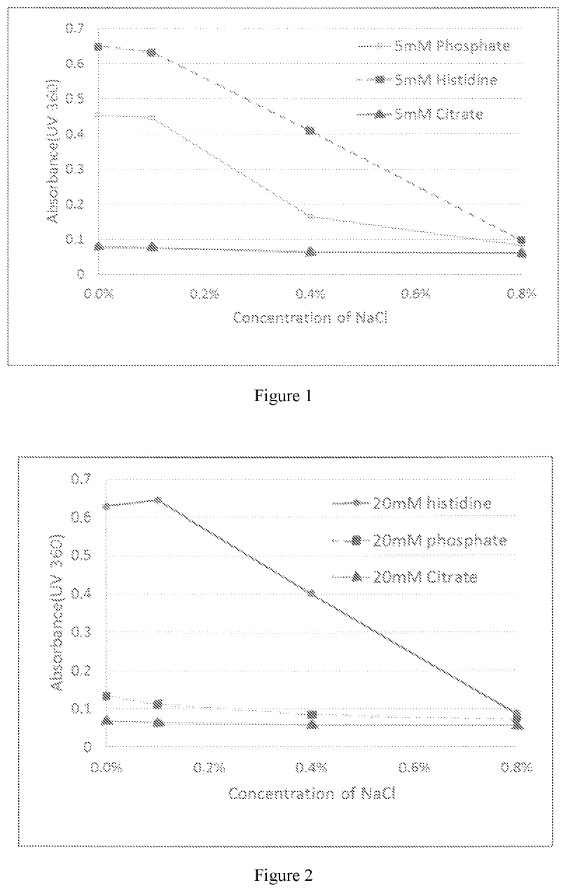

The results were shown in FIGS. 1 and 2, demonstrating that the citrate buffer used in the pharmaceutical composition comprising aFGF provides a better effect at preventing precipitation than other buffers, i.e., phosphate and histidine buffers.

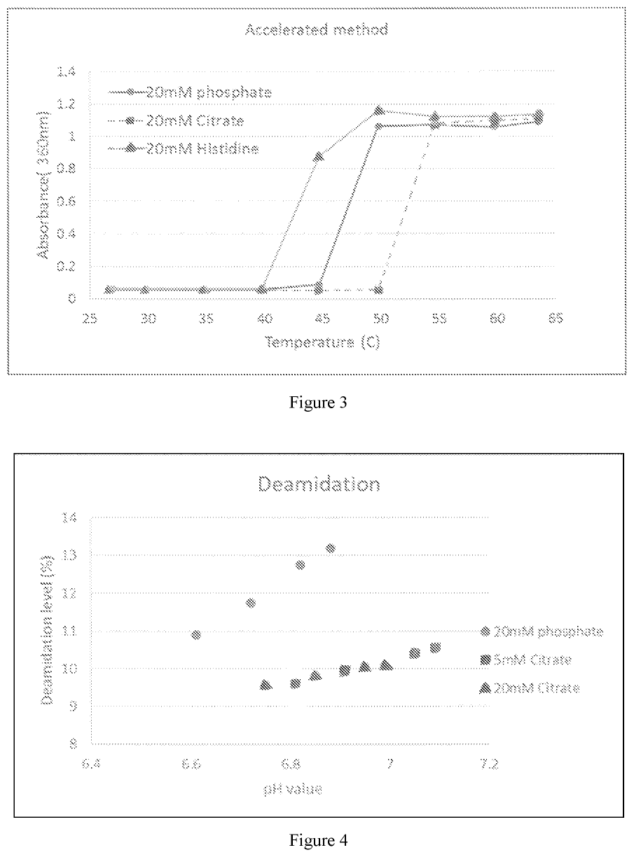

An accelerated method was established in Test 1. It was shown in FIG. 3 that the UV 360 nm absorption of test samples increased rapidly in a narrow range of temperature. The accelerated method could indicate the denaturation of ES135, and it's useful to differentiate the stability of formulations not tending to precipitate in a few days. The accelerated method indicated the citrate buffer was the best buffer for ES135, and the result was consistent with the results shown in FIGS. 1 and 2.

### Example 2: Effects of Different Salts on Protein Deamidation

The deamidation level in Test 1 was determined by HPIEC. As shown in FIG. 4, the deamidation level was correlated with the pH value; however, the phosphate buffer caused the higher deamidation level than the citrate buffer at corresponding pH value.

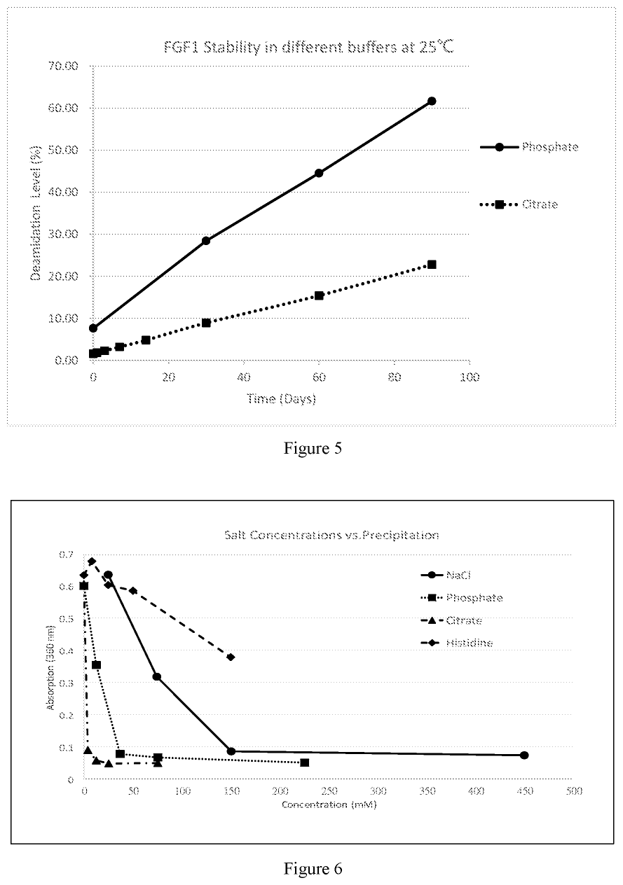

The time-course deamidation level was measured for 20 mM phosphate buffer and 30 mM citrate buffer. As shown in FIG. 5, the phosphate buffer caused the higher deamidation level than the citrate buffer at corresponding time points from 0 to 90 days.

### Example 3: Stabilizing Effect of Different Buffers

According to the invention, the pharmaceutical composition may be prepared in the form of a liquid formulation. In Test 2, the stabilization effects of the three buffers and NaCl were tested in a range of concentration (FIG. 6). NaCl salts, as well as phosphate and citrate ions, could provide an efficacy in stabilizing the ES135 by preventing the precipitation. The citrate buffer also exhibited the best stabilization effect on preventing ES135 from precipitation. If the boundary line of precipitation was set up in 0.1 Abs, the corresponding concentrations of citrate, phosphate, and NaCl were about 4 mM, 37 mM, and 151 mM respectively. Therefore, the citrate buffer at a concentration of 5 mM appeared to have an equivalent ability to 150 mM NaCl on preventing the precipitation.

### Example 4: Stabilizing Effects of Citrate Buffer with Different pH Values

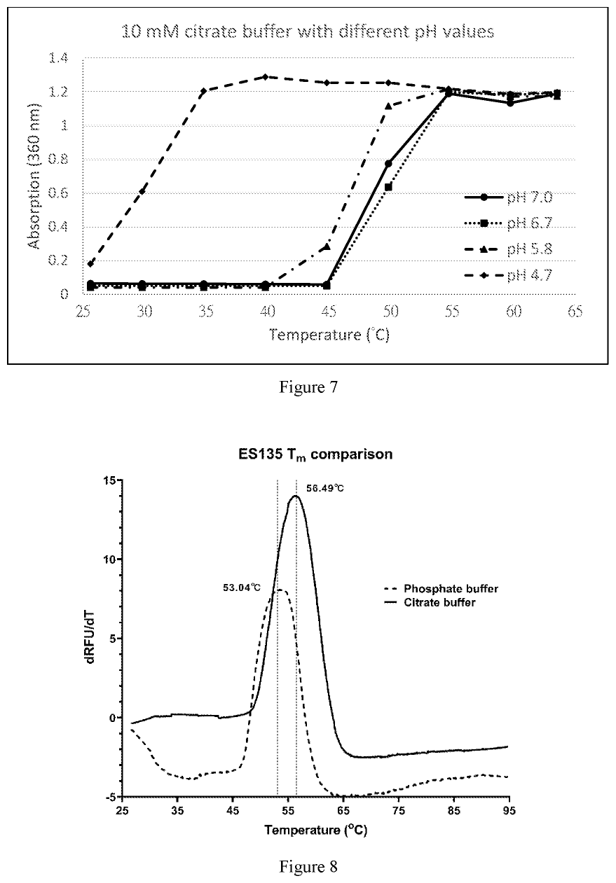

As shown in Table 6, the pH values ranging between 4.7˜7.0 were evaluated by the accelerated method, and the result was shown in FIG. 7, demonstrating that ES135 in 10 mM citrate buffer had similar stability within pH 5.8˜7.0, and started to precipitate beyond 45° C. However, ES135 was very unstable at pH 4.7 and started to precipitate at 25° C., which was much lower than 45° C.

TABLE 6
Effects of different pH values on protein precipitation
Test 3

| Factors | Buffer | pH value |
|---|---|---|
| | 10 mM citrate buffer | 7.0 |
| | | 6.7 |
| | | 5.8 |
| | | 4.7 |

Testing methods: Accelerated method

### Example 5: Tm Values of ES135 in Different Buffers

The melting temperature (Tm) of the ES135 in phosphate buffer and citrate buffer was measured based on the method described below. As shown in FIG. 8, Tm value of ES135 in phosphate buffer was 53.04° C., however Tm value of ES135 in citrate buffer was 56.49° C. This result indicates that ES135 in citrate buffer obviously possessed a higher stability.

### Example 6: Formulations of the Lyophilized Product

The stabilizers and bulking agents are crucial for the lyophilized drug product. The stabilizers such as carbohydrates can stabilize protein by providing —OH group in the surrounding area while the water is moved from the liquid formulation. The bulking agents such as mannitol could support the structure of lyophilization drug product. Two stabilizers (trehalose and sucrose) and one bulking agent (mannitol) were tested for the lyophilization of ES135 formulation. The 4 representative examples of lyophilized formulation are listed in Table 7. Detailed procedure for making lyophilized formulation is described in Methods.

TABLE 7
Representative examples of lyophilized formations

| | | aFGF | Citrate | Mannitol | Trehalose | Sucrose |
|---|---|---|---|---|---|---|
| # | pH | (mg/ml) | (mM) | (mg/ml) | (mg/ml) | (mg/ml) |
| 1 | 6.6 | 1 | 5 | 0 | 50 | 0 |
| 2 | 6.6 | 1 | 5 | 15 | 50 | 0 |
| 3 | 6.6 | 2 | 10 | 15 | 50 | 0 |
| 4 | 6.6 | 1 | 5 | 0 | 0 | 50 |

In conclusion, with the addition of citric acid compound as buffer system, the stability of the pharmaceutical compositions comprising aFGF is significantly improved. Also, the pharmaceutical compositions of the present invention can be further processed into lyophilized form.

While the foregoing written description of the invention enables one of ordinary skill in the art to make and use what is considered presently to be the best mode thereof, those of ordinary skill in the art will understand and appreciate the existence of variations, combinations, and equivalents of the specific embodiments, methods, and examples herein. The invention should therefore not be limited by the above described embodiments, methods, and examples, but by all embodiments and methods within the scope and spirit of the invention.

What is claimed is:

1. A pharmaceutical composition with improved stability comprising acidic fibroblast growth factor (aFGF) and a citric acid compound.

2. The pharmaceutical composition according to claim 1, wherein the citric acid compound is citric acid or isocitric acid.

3. The pharmaceutical composition according to claim 1, wherein the pharmaceutical composition is in the form of a liquid formulation.

4. The pharmaceutical composition according to claim 3, wherein the concentration for the citric acid compound in the liquid formulation ranges from 5 mM to 75 mM.

5. The pharmaceutical composition according to claim 3, wherein the liquid formulation has a pH value within the range of pH 5.8 to pH 7.0.

6. The pharmaceutical composition according to claim 1, wherein aFGF is the protein having an amino acid sequence at least 70%, 75%, 80%, 85%, 90%, 95%, or 98% identical to the amino acid sequence of SEQ ID NO: 1.

7. The pharmaceutical composition according to claim 1, wherein aFGF is the protein consisting of an amino acid sequence of SEQ ID NO: 1.

8. The pharmaceutical composition according claim 1, wherein the composition is administered subcutaneously, topically, intranasally, intravenously, intramuscularly, intraneurally, intraperitoneally, intracerebroventricularly or intrathecally.

9. The pharmaceutical composition according to claim 1, wherein the pharmaceutical composition is in the form of a lyophilized formulation.

10. The pharmaceutical composition according to claim 9, further comprising mannitol, sugar, or the combination thereof.

11. The pharmaceutical composition according to claim 10, wherein the sugar is selected from the group consisting of trehalose, sucrose, and the combination thereof.

12. A use of a citrate buffer in improving the stability of a pharmaceutical composition comprising aFGF.

13. A method for stabilization of a pharmaceutical composition comprising aFGF comprising mixing aFGF with a citrate buffer to obtain a liquid formulation.

14. The method according to claim 13, which further comprises the step of lyophilizing the liquid formulation.


SEQUENCE LISTING (1 sequence)

SEQ ID NO: 1 - protein, 135 aa, Artificial Sequence
  Peptide derived from Homo sapiens Acid- Fibroblast Growth Factor (a-FGF)
ANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD